(19) United States
(12) Patent Application Publication
(10) Pub. No.: US 2006/0171902 A1
(43) Pub. Date: Aug. 3, 2006

(54) Engineered oral tissue structural constructs

(76) Inventors: Anthony Atala, Winston Salem, NC (US); Akira Joraku, Winston-Salem, NC (US); Christopher A. Sullivan, Winston-Salem, NC (US); James Yoo, Winston Salem, NC (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/048,097

(22) Filed: Feb. 1, 2005

(51) Int. Cl. A61K 8/96; A61K 35/12

(52) U.S. Cl. 424/50; 424/93.7

(57) ABSTRACT

The invention is directed to compositions and methods for preparing an artificial oral tissue. The artificial oral tissue is prepared using a biocompatible substrate seeded with salivary gland cells that develop to produce a salivary gland tissue layer with a prototypal salivary system and a prototypal secretory system. The salivary gland cells proliferate, mature and differentiate into a salivary gland structure that are analogous to its in vivo counterpart.

### BACKGROUND OF THE INVENTION

The technical field of this invention relates to the construction of artificial oral tissue structures by seeding cultured cell populations of oral cells, e.g., salivary gland cells onto or into a biocompatible substrate. The invention is particularly useful in constructing artificial salivary glands.

Radiation therapy for head and neck cancer results in atrophy, fibrosis and degeneration of major and minor salivary gland tissue leads to salivary gland hypofunction and xerostomia. This condition affects approximately forty thousand new patients annually in the United States and as many as 500,000 people worldwide. The growing trend toward the use of organ sparing chemoradiation therapy for most oropharyngeal and laryngeal cancers predicts a growing population afflicted with xerostomia that hinders speech, dental health, swallowing, nutrition and general quality of life.

Current management of radiation induced xerostomia includes the administration of saliva substitutes and sialogogues (Hamlar et al. (1996) Laryngoscope 106:972-976; Warde et al. (2002) Int J Radiat Oncol Biol Phys 54:9-13; and Haddad et al. (2002) Radiother Oncol 64:29-32). Gel or spray saliva substitutes have been used in order to lubricate the oral cavity; however, the effect of these medications is transient and necessitates frequent administration. Oral sialogogues such as pilocarpine hydrochloride and cevimeline hydrochloride have been used with some success to stimulate existing hypofunctioning salivary glands, but the systemic side effects of these medications can be intolerable to some individuals. Recently, neomorphic strategies have been proposed, including adenoviral gene transfer of a water channel protein. in one such study, investigators successfully demonstrated the transformation of ductal epithelium into acinar cells in rats with a measurable increase in saliva production by the transfected tissue (Delporte et al. (1997) Proc Natl Acad Sci U S A 94:3268-3273). It is, however, unknown whether irradiated target ductal epithelium will transform into acinar cells in a human clinical model.

Engineering new tissues from cultured cells represents another new approach to treat patients suffering from the loss or malfunction of certain tissues (See e.g., Atala et al., U.S. Pat. No. 6,576,019; U.S. Pat. No. 6,547,719; U.S. Pat. No. 6,479,064; and U.S. Pat. No. 6,428,802). However, with the limited exception of oral mucosa, the techniques of cell and tissue culture have not been successfully applied in the engineering of oral tissues. Current cell culture techniques, such as those used in the regeneration of skin, and even oral mucosa, are not transferable to the regeneration of other oral tissues as the existing techniques produce epithelia which require an appropriate connective tissue bed in vivo for successful grafting. The art therefore lacks appropriate techniques for the production of oral tissues ex vivo that will repair and regenerate specific oral tissues in vivo.

Therefore, a need exists for reconstructing artificial oral tissue, in particular, the creation of functional salivary glands composed of a patient's own glandular cells, to provide a physiologic solution to salivary gland hypofunction.

### SUMMARY OF THE INVENTION

The present invention provides methods and compositions for preparing artificial oral tissue using a biocompatible substrate seeded with oral tissue cells. The biocompatible substrate is seeded with a population of cultured oral tissue cells e.g., salivary gland cells, which attach to the biocompatible substrate and develop into an oral tissue layer. Continued growth and differentiation of the oral tissue layer on the biocompatible substrate results in the formation of oral tissue structures, such as salivary glands that function as native in vivo oral tissue structures.

Accordingly, in one aspect, the invention pertains to a method of preparing an artificial oral tissue construct. The method involves seeding a population of cultured oral tissue cells onto or into a substrate, preferably a biocompatible substrate, such that the oral tissue cells attach to the biocompatible substrate. The oral tissue cells are cultured onto or into the substrate until the oral tissue cells produce an oral tissue layer comprising a primitive salivary system that can produce saliva. In addition to the primitive salivary system, the artificial construct may also comprise a primitive secretory system that can secrete the saliva from the cells.

The methods and compositions of the invention can be used to create artificial oral tissue structures such as artificial oral glands or ducts. Examples of such glands and ducts include, but are not limited to, salivary glands, submandular glands, sublingual glands, lingual glands, labial glands, buccal glands, palatine glands, striated ducts, and excretory ducts.

The artificial oral tissue construct can be prepared by isolating the appropriate cell population from a subject, e.g., a human subject. Preferred oral tissue cells are human salivary gland cells.

In another aspect, the invention pertains to a method of preparing an artificial salivary gland construct by seeding a population of cultured salivary gland cells onto or into a substrate, preferably a biocompatible substrate, such that the salivary gland cells attach to the biocompatible substrate. These salivary gland cells are cultured in the substrate until the salivary gland cells produce a salivary gland tissue layer comprising a primitive salivary system that produces saliva. The salivary gland construct can further comprise a primitive secretory system that secretes the saliva.

In another aspect, the invention pertains to an artificial oral tissue construct comprising a substrate, preferably a biocompatible substrate seeded with a population of cultured oral tissue cells. The oral tissue cells attach to the biocompatible substrate to produce an oral tissue layer comprising a primitive salivary system that produces saliva. The oral structural construct may further comprise a primitive secretory system that secretes the saliva.

In another aspect, the invention pertains to an artificial salivary gland construct comprising a substrate, preferably a biocompatible substrate seeded with a population of cultured salivary gland cells The salivary gland cells attach to the biocompatible substrate to produce a salivary gland tissue layer comprising a primitive salivary system that produces saliva. The salivary gland construct may further comprise a primitive secretory system that secretes the saliva.

The methods and compositions of the invention may be used to ameliorate or treat a number of oral disorders. Accordingly, in another aspect, the invention pertains to a method of ameliorating an oral disorder in a subject by implanting a biocompatible substrate seeded with a population of cultured oral tissue cells. The oral tissue cells attach to the biocompatible substrate to produce an oral tissue layer comprising a primitive salivary system and a primitive secretory system. As the implanted construct further develops, the subject is monitored for the amelioration in the oral disorder.

Oral disorders that can be ameliorated with the methods and compositions of the invention include, but are not limited to, conditions that arise due to salivary gland tumors, cystic fibrosis, Sjögren's syndrome, sialoadenitis, parotitis, sialoangitis, sialodochitis, sialolithiasis, sialodocholithiasis, mucocele, ranula, hyposecretion, ptyalism, sialorrhea, xerostomia, benign lymphoepithelial lesion of salivary gland; sialectasia; sialosis; stenosis of salivary duct; and stricture of salivary duct.

In yet another aspect, the invention pertains to a method of ameliorating xerostomia by implanting a biocompatible substrate seeded with a population of cultured salivary gland cells. The salivary gland cells attach to the biocompatible substrate to produce a salivary gland tissue layer comprising a primitive salivary system and a primitive exectory system. As the implanted construct develops, the subject is monitored for the amelioration of xerostomia.

### DESCRIPTION OF THE DRAWINGS

FIG. 1 is a photograph of a Western blot analysis of primary salivary gland cells and implants with cells showing expression of α-amylase (61 kDa), aquaporin 5 (AQP5, 29 kDa), and cytokeratins (CK) AE1/AE3 (45 kDa), that is similar to normal human salivary gland tissue;

FIG. 2 is a photograph of an agarose gel of an RT-PCR reaction with the expected size of 474 bp (α-amylase) and 225 bp (aquaporin 5) obtained with RNA form primary human salivary glands, implants with cells, and normal human salivary gland tissue; and

FIG. 3 is a bar graph showing the high levels of α-amylase activity of the implanted scaffolds. The implants without salivary gland cells and host subcutaneous tissue showed base levels of activity.

### DETAILED DESCRIPTION

So that the invention may more readily be understood, certain terms are first defined:

The term “attach” or “attaches” as used herein refers to cells adhered directly to the biocompatible substrate or to cells that are themselves attached to other cells.

The phrase “biocompatible substrate” as used herein refers to a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of an structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

The phrases “prototypal salivary system” or “primitive salivary system” are used interchangeably herein and refer to the early stages of development of a salivary system that is capable of producing saliva, i.e., the first of a functional kind. The primitive salivary system includes salivary proteins such as aquaporinin-5, and digestive enzymes such as alpha-amylase (α-amylase). A number of multifunctional proteins and enzymes exist in the saliva which function as anti-bacterial agents, anti-fungal agents, anti-viral agents, buffering agents, aid in digestion, lubrication and viscosity. Thus, the primitive salivary system is capable of producing at least one of the proteins or enzymes. For example, the primitive salivary system can produce at least one digestive enzyme which includes, but is not limited to, amylases, mucins, and lipases. In one embodiment, the primitive salivary system produces α-amylase. The primitive salivary system may also produce buffering agents such as carbonic anhydrases and histatins; mineralization agents such as cystatins, histatins, proline rich proteins, and statherins; lubricating and visco-elasticity agents such as mucins and statherins; tissue coating agents such as amylases, cystatins, mucins, proline-rich proteins and statherins; antifungal agents such as histatins; anti-viral agents such as cystatins and mucins; and anti-bacterial agents such as amylases, lysozyme, cystatins, histatins, mucins, and peroxidases.

The phrases “prototypal secretory system” or “primitive secretory system” are used interchangeably herein and refer to the early stages of development of a secretory system, that is capable of secreting saliva, proteins and enzymes into the mouth.

The phrase “oral tissue cells” refers to any cell population derived from the mouth. These include one or more different cells types that can be isolated from the salivary glands, submandular gland, sublingual gland, lingual glands, labial glands, buccal glands, palatine glands, striated ducts, excretory ducts, dental pulp tissue, dentin, periodontium, bone, cementum, gingival submucosa, oral submucosa, tongue and taste bud tissues. In a preferred embodiment, the oral tissue cells are derived from the salivary gland. Examples of oral tissue cells include, but are not limited to, myoepithelial cells, epithelial cells, and the like.

The phrase “oral tissue” refers to any aggregate of cells that forms a structure in the mouth. By way of example only, oral tissue includes salivary glands, submandular gland, sublingual gland, lingual glands, labial glands, buccal glands, palatine glands, striated ducts, excretory ducts, dental pulp tissue, dentin, periodontium, bone, cementum, gingival submucosa, oral submucosa, tongue and taste bud tissues. In a preferred embodiment, the oral tissue is a salivary gland. The phrase also refers to a part of the oral tissue, e.g., a part of the salivary gland.

The phrase “oral tissue construct” refers to a substrate, preferably a biocompatible substrate that has been seeded with oral tissue cells in which the cells have attached, grown, proliferated, differentiated and populated the biocompatible substrate. This phrase also refers to a neomorphic structure representing the early stages of development of the oral tissue.

The phrase “salivary gland construct” refers to a substrate, preferably biocompatible substrate that has been seeded with salivary gland cells in which the cells have attached, grown, proliferated, differentiated, and populated the biocompatible substrate. This phrase also refers to the neomorphic structure representing the early stages of development of the salivary gland.

The phrase “oral disorder” refers to diseases or disorders that affect the mouth. In particular, diseases or disorders that effect the production of saliva. Examples of oral disorders include, but are not limited to, salivary gland tumors, cystic fibrosis, Sjögren's syndrome, sialoadenitis, parotitis, sialoangitis, sialodochitis, sialolithiasis, sialodocholithiasis, mucocele, ranula, hyposecretion, ptyalism, sialorrhea, xerostomia, benign lymphoepithelial lesion of salivary gland; sialectasia; sialosis; stenosis of salivary duct; and stricture of salivary duct.

The term “subject” as used herein is intended to include living organisms in which an immune response is elicited. Preferred subjects are mammals. Examples of subjects include but are not limited to, humans, monkeys, dogs, cats, mice, rates, cows, horses, pigs, goats and sheep.

The term “ameliorate” as used herein refers to an improvement or change in a condition associated with an oral disorder. For example, an improvement in xerostomania, which can be monitored by measuring increased saliva production.

I Structure of Oral Tissues

The invention pertains to methods and compositions for producing and using artificial oral tissue for repair or replacement. In particular, the invention pertains to methods of producing artificial oral glands such as salivary glands. The major glands in the mouth and neck are paired and have long ducts. There are three major paired salivary glands: the submandibular, the sublingual and the parotid glands. They differ from one another in the relative abundance of serous and mucous acini, and in the length of the various kinds of ducts. The minor salivary glands are located in the submucosa of different parts of the oral cavity.

(i) Major glands:

(a) Salivary (Parotid) Gland

The salivary gland, also known as the parotid gland, originates in the ectoderm, developing oral cavity epithelium. Its function is to secrete saliva. Saliva functions to moisten dry foods to aid swallowing, provides a medium for dissolved and suspended food materials that chemically stimulate taste buds, buffers the contents of the oral cavity through its high concentration of bicarbonate ion, digests carbohydrates by the digestive enzyme α-amylase, controls the bacterial flora due to the presence of the antibacterial enzyme lysozyme, and also provides a source of calcium and phosphate ions essential for normal tooth maintenance.

The parotid gland is located subcutaneously, below and in front of ear. The gland is composed of capsules of moderately dense connective tissue with septa, loose connective tissue and white adipose tissue between the secretory acini, blood vessels, facial nerve (cranial nerve VII). The secretory acini (portions) are spherical and are organized into lobules and lobes. The parotid gland has serous (epithelial) cells, that function to secrete proteins. The parotid gland also has myoepithelial cells located between the serous cells and the basal lamina of epithelium. These myoepithelial cells function to move the secretory products toward the excretory duct by contraction.

The parotid gland also has several ducts. The intercalated ducts are lined by low cuboidal epithelial cells. These intercalated ducts function as conduits for the secretory products, secrete bicarbonate ion into the acinar product, and absorb chloride ion from the acinar product. Striated ducts are lined by simple cuboidal or columnar epithelium. These striated ducts function as conduits for the secretory products, reabsorb sodium from the primary secretion, and add potassium to the secretion. Excretory ducts are lined by stratified cuboidal or pseudostratified columnar epithelium. These are the principal duct (Stensen's duct) and travel in the connective tissue of the face and enter the oral cavity opposite the second upper molar tooth.

(b) Submandular Gland

The submandular gland is located under the floor of the mouth, close to the mandible. The gland is composed of capsules of moderately dense connective tissue with septa, loose connective tissue between the secretory acini, blood vessels, nerves. The secretory acini (portions) are predominantly spherical and are organized into lobules and lobes. The submandular gland is predominantly composed of serous (epithelial) cells that function to secrete proteins. The submandular gland also has mucous (epithelial) cells which secrete mucin. Myoepithelial cells are also present in the submandular gland and are located between the serous cells and the basal lamina of epithelium. These cells move the secretory products toward the excretory duct by contraction.

The cells of the acini appear triangular in sections, with their apex directed toward the lumen, and their base resting on a basement membrane. They secrete their product in a merocrine fashion into the lumen. Contractile cells called myoepithelial cells or basket cells lie between the basement membrane and the plasma membrane of the secretory cells. They are also found in the proximal part of the duct system. Myoepithelial cells posess many actin-containing microfilaments, which squeeze on the secretory cells and move their products toward the excretory ducts.

Acini can be either serous or mucous. The secretion of serous cells is thin, watery and proteinaceous. Serous cells have a rounded nucleus and secretory granules in their cytoplasm. They are joined near their apical surfaces by junctional complexes. Mucous cells secrete a viscous, glycoprotein-rich product, which is stored as mucinogen granules. The nuclei are typically flattened against the base of the cells (unless the cells have just discharged their contents, in which case they look more like serous cells). Mucous cells typically look pale and empty in standard histological sections, because their granules are lost during preparation. The submandibular gland of humans is predominantly serous. Its mucous acini are quite frequently capped with a serous demilune, a crescent of serous cells around one or more of their surfaces. Because of small size of the lumen of acini and the variability in sectioning, lunina are rarely seen in the acini.

The submandular gland also has intercalated ducts which are lined by low cuboidal epithelial cells. There are three types of ducts in the submandibular gland: intercalated ducts, secretory ducts (also known as striated ducts), and excretory ducts. Intercalated ducts are slender ducts continuous with the terminal acini, and lined with flat, spindle-shaped cells. They secrete bicarbonate ion into and absorb chloride ion from the acinar product. Secretory ducts have eosinophilic cuboidal to columnar cells with basal striations. These result from infoldings of the basal membranes in which are found many mitochondria. Secretory ducts resorb sodium and secrete potassium. As they approach the excretory ducts, their diameter may exceed that of the acini. Both intercalated and secretory ducts are found within the parenchyma of the gland and are therefore intralobular ducts.

The largest ducts are the excretory ducts. They are found in the connective tissue septa, and are therefore interlobular ducts. They ultimately connect with the oral cavity. Their epithelium is variable, it can be simple cuboidal, stratified cuboidal, stratified columnar or pseudostratified. Near the oral cavity, it becomes stratified squamous. Excretory ducts do not change the secretory product.

(c) Sublingual Gland

The sublingual gland is located in the floor of the mouth anterior to the submandibular gland. The gland is composed of moderately dense connective tissue with septa, loose connective tissue between the secretory acini, blood vessels, nerves. The secretory acini (portions), the mucous secretory units, may be more tubular than purely acinar and are organized into lobules. The sublingual gland is predominantly composed of mucous (epithelial) cells which secrete mucin. These glands also have serous (epithelial) cells that secrete protein. Myoepithelial cells are also present in the sublingual gland and are located between the serous cells and the basal lamina of epithelium. These cells move the secretory products toward the excretory duct by contraction.

The ducts of the submandibular gland are lined by columnar epithelium or pseudostratified columnar epithelium and empty into the submandibular duct as well as directly onto the floor of the mouth.

(ii) Minor Glands

Minor glands are simple branched tubular and acinar glands in the submucosa of oral cavity. These include lingual glands, labial glands, buccal glands, and palatine glands.

Of all the organs in the craniofacial-oral-dental complex, the salivary glands and their secretory product, saliva, form the strongest link between oral and systemic health. Salivary function is sensitive to changes in a subject's general well-being, ranging from subtle effects of over-the-counter cold medications to the devastation of life-threatening disease.

With its vast antimicrobial arsenal, saliva represents an evolutionary selective advantage for the host against invading pathogens such as HIV, the fungus Candida albicans, and a host of bacteria associated with oral and systemic diseases. Secretory antibodies, for example, directed against viral pathogens such as poliovirus and cold viruses, as well as the anti-HIV agent SLPI, are found in saliva. Large salivary glycoproteins called mucins appear to have antiviral properties as do cystatins, a family of cysteine-rich proteins that are active against herpes viruses.

Saliva also contains histatins, anti-fungal proteins that are potent inhibitors of candida, which is normally kept in check at extremely low levels in the mouth. When the oral balance is upset, however, by HIV infection or other immunosuppressive and debilitating disorders, anti-fungal defenses are overwhelmed and candida flourishes uncontrolled. Reinforcing saliva's antiviral and anti-fungal activity are salivary constituents that thwart bacterial attack. These enzymes destroy the opposition by various mechanisms, including degrading bacterial membranes, inhibiting the growth and metabolism of certain bacteria, and disrupting vital bacterial enzyme systems.

Functioning in concert, these and other protective factors in saliva help to maintain the oral environment in optimal working order and restore it to more normal conditions when disturbed.

On the basis of the weight of the glands producing it, the volume of saliva exceeds that of other digestive organs by as much as 40 times. Saliva moistens the oral mucosa as well as dry food before swallowing. Its high bicarbonate content buffers the oral cavity. It provides a medium for food materials to stimulate taste buds. It begins the digestion of carbohydrates via the digestive enzyme amylase. It controls the bacterial flora by secreting lysozyme. In the absence of saliva, infections and caries develop in the oral tissues. The salivary glands also secrete IgA and potassium, and resorb sodium.

The invention also pertains to generating smaller units of an oral tissue, for example smaller units of a salivary gland tissue such as an ancinar structures from human salivary gland epithelial cells (See Examples). These smaller units can be generated within days in vitro within a biocompatible substrate, such as a three-dimensional gel substrate. These smaller units can then be implanted in vivo and used to reconstitute a salivary gland.

II Diseases Affecting Salivary Glands

Salivary gland dysfunction can lead to a number of oral disorders. The parotid, submandibular, and sublingual glands that comprise the major salivary glands are directly affected by a variety of conditions, including infection (such as mumps), obstructions, developmental disorders, and tumors. Major diseases, such as salivary gland tumors, cystic fibrosis (CF) and Sjögren's syndrome, can devastate these vital glands. The methods and compositions of the invention can be used to treat several diseases and disorders associated with reduced or non-existent salivary gland function. These disorders include, but are not limited to, sialoadenitis (parotitis, sialoangitis, sialodochitis); sialolithiasis (calculus of salivary gland or duct, stone of salivary gland or duct, sialodocholithiasis); mucocele (extravasation cyst of salivary gland, ranula); disturbance of salivary secretion (hyposecretion, ptyalism, sialorrhea, xerostomia); benign lymphoepithelial lesion of salivary gland; sialectasia; sialosis; stenosis of salivary duct; and stricture of salivary duct. The methods and compositions of the invention are particularly useful for ameliorating salivary gland dysfunction arising due to the following disorders:

(i) Salivary Gland Tumors

Salivary gland tumors (parotid gland tumor, submandibular gland tumor, sublingual gland tumor, oral cancer) require surgery, radiation therapy, chemotherapy, reconstructive surgery, or a combination of these. While head and neck radiation treatment kills cancerous cells, it also often destroys vital acinar cells that lie within the radiation field. Patients are unable to produce adequate saliva, leading to a host of long-term problems including xerostomia (dry mouth), mucositis, rampant dental caries, infections of the mouth and pharynx, and difficulty with swallowing, speech and taste. These conditions dramatically reduce quality of life and can also be the source of systemic infections that may threaten patient survival or interfere with their cancer treatment.

(ii) Xerostomia

Xerostomia arises due to radiation treatment. The lack of saliva production leads to increased risk of infections. Another major source of dry mouth is a result of medication. More than 400 prescription and over-the-counter drugs are known to have xerostomic effects. Many of these medications are taken daily, to treat chronic conditions such as hypertension and depression. Although salivary gland function does not normally decline with age, the oral dryness experienced by many older persons from certain diseases and long-term medications heightens their risk for oral and dental infections.

(iii) Sjögren's Syndrome

Sjögren's syndrome, an autoimmune disorder that primarily affects women. Classic symptoms include dry mouth, eyes and other mucosal surfaces, accompanied in about half the cases by a connective tissue disease such as rheumatoid arthritis or systemic lupus erythematosus. The oral dryness interferes with normal functions of talking, chewing and swallowing and, deprived of the protective properties of saliva, puts Sjögren's syndrome patients at high risk for dental and oral infections. The methods an compositions of the invention can be used to alteration salivary gland function associated with Sjögren's syndrome.

(iv) Cystic Fibrosis

In cystic fibrosis, a defect in chloride ion transport causes exocrine gland secretions, including saliva, to be thick and viscid and leads to chronic lung disease and pancreatic insufficiency. Studies of salivary acinar (salt and water secreting) cells, a convenient model for exploring mechanisms of chloride ion transport, have greatly expanded the understanding of exocrine gland transport systems in human salivary glands. The identification of the defective gene in cystic fibrosis has also led to clinical trials using gene therapy to treat this disorder.

III Substrates

The invention pertains to generating artificial oral tissue structures. This is accomplished by seeding cultured oral tissue cells onto or into a substrate. The substrate is preferably a biocompatible substrate. Biocompatible refers to materials that do not have toxic or injurious effects on biological functions. Biodegradable refers to material that can be absorbed or degraded in a patient's body. Representative materials for forming the biodegradable material include natural or synthetic polymers, such as, collagen, poly(alpha esters) such as poly(lactate acid), poly(glycolic acid), polyorthoesters and polyanhydrides and their copolymers, which degraded by hydrolysis at a controlled rate and are reabsorbed. These materials provide the maximum control of degradability, manageability, size and configuration. Preferred biodegradable polymer materials include polyglycolic acid and polyglactin, developed as absorbable synthetic suture material.

Polyglycolic acid and polyglactin fibers may be used as supplied by the manufacturer. Other biodegradable materials include, but are not limited to, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends of these materials. The material may be impregnated with suitable antimicrobial agents and may be colored by a color additive to improve visibility and to aid in surgical procedures.

In some embodiments, attachment of the cells to the substrate is enhanced by coating the substrate with compounds such as basement membrane components, agar, agarose, gelatin, gum arabic, collagens, fibronectin, laminin, glycosaminoglycans, mixtures thereof, and other materials having properties similar to biological matrix molecules known to those skilled in the art of cell culture. Mechanical and biochemical parameters ensure the substrate provide adequate support for the cells with subsequent growth and proliferation. Factors, including nutrients, growth factors, inducers of differentiation or dedifferentiation, products of secretion, immunomodulators, inhibitors of inflammation, regression factors, biologically active compounds which enhance or allow ingrowth of the lymphatic network or nerve fibers, and drugs, can be incorporated into the substrate or provided in conjunction with the substrate. Similarly, polymers containing peptides such as the attachment peptide RGD (Arg-Gly-Asp) can be synthesized for use in forming matrices.

Coating refers to coating or permeating a substrate with a material such as, liquefied copolymers (poly-DL-lactide co-glycolide 50:50 80 mg/ml methylene chloride) to alter its mechanical properties. Coating may be performed in one layer, or multiple layers until the desired mechanical properties are achieved. These shaping techniques may be employed in combination, for example, a polymeric matrix can be weaved, compression molded and glued together. Furthermore different polymeric materials shaped by different processes may be joined together to form a composite shape. The composite shape can be a laminar structure. For example, a polymeric matrix may be attached to one or more polymeric matrixes to form a multilayer polymeric matrix structure. The attachment may be performed by any suitable means such as gluing with a liquid polymer, stapling, suturing, or a combination of these methods. In addition, the polymeric matrix may be formed as a solid block and shaped by laser or other standard machining techniques to its desired final form. Laser shaping refers to the process of removing materials using a laser.

The polymers can be characterized for mechanical properties such as tensile strength using an Instron tester, for polymer molecular weight by gel permeation chromatography (GPC), glass transition temperature by differential scanning calorimetry (DSC) and bond structure by infrared (IR) spectroscopy; with respect to toxicology by initial screening tests involving Ames assays and in vitro teratogenicity assays, and implantation studies in animals for immunogenicity, inflammation, release and degradation studies. In vitro cell attachment and viability can be assessed using scanning electron microscopy, histology, and quantitative assessment with radioisotopes.

Substrates can be treated with additives or drugs prior to implantation (before or after the polymeric substrate is seeded with cells), e.g., to promote the formation of new tissue after implantation. Thus, for example, growth factors, cytokines, extracellular matrix components, and other bioactive materials can be added to the substrate to promote graft healing and formation of new tissue. Such additives will in general be selected according to the tissue or organ being reconstructed or augmented, to ensure that appropriate new tissue is formed in the engrafted organ or tissue (for examples of such additives for use in promoting bone healing, see, e.g., Kirker-Head, C. A. Vet. Surg. 24 (5): 408-19 (1995)). For example, vascular endothelial growth factor (VEGF, see, e.g., U.S. Pat. No. 5,654,273 herein incorporated by reference) can be employed to promote the formation of new vascular tissue. Growth factors and other additives (e.g., epidermal growth factor (EGF), heparin-binding epidermal-like growth factor (HBGF), fibroblast growth factor (FGF), cytokines, genes, proteins, and the like) can be added in amounts in excess of any amount of such growth factors (if any) which may be produced by the cells seeded on the substrate. Such additives are preferably provided in an amount sufficient to promote the formation of new tissue of a type appropriate to the tissue or organ, which is to be repaired or augmented (e.g., by causing or accelerating infiltration of host cells into the graft). Other useful additives include antibacterial agents such as antibiotics.

The biocompatible polymer may be shaped to produce a substrate using methods such as, solvent casting, compression molding, filament drawing, meshing, leaching, weaving and coating. In solvent casting, a solution of one or more polymers in an appropriate solvent, such as methylene chloride, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the oral tissue. Next a solvent is used to dissolve away one of the components, resulting in pore formation. (See Mikos, U.S. Pat. No. 5,514,378, hereby incorporated by reference).

In nucleation, thin films in the shape of the oral tissue is exposed to radioactive fission products that create tracks of radiation damaged material. Next, the polycarbonate sheets are etched with acid or base, turning the tracks of radiation-damaged material into pores. Finally, a laser may be used to shape and bum individual holes through many materials to form an oral tissue structure with uniform pore sizes. The polymeric substrate can be fabricated to have a controlled pore structure that allows nutrients from the culture medium to reach the deposited cell population. In vitro cell attachment and cell viability can be assessed using scanning electron microscopy, histology and quantitative assessment with radioisotopes.

Thus, the polymeric substrates can be shaped into any number of desirable configurations to satisfy any number of overall system, geometry or space restrictions. The polymeric substrates can be shaped to different sizes to conform to the oral structures, e.g., salivary glands of different sized patients.

The substrate may also be a biocompatible gel such as a collagen gel that can be used to form a three-dimensional substrate. The cells can be mixed into the gel before the gel is solidified. These three dimensional substrates provide an environment that encourages cells growth in all directions.

IV Culturing Cells

The artificial oral tissue, e.g., a salivary gland, can be created by using allogenic cell populations derived from the subject's own mouth. The artificial oral tissue can also be xenogenic, where cell populations are derived from a mammalian species that are different from the subject. For example, oral tissue cells can be derived from mammals such as monkeys, dogs, cats, mice, rats, cows, horses, pigs, goats and sheep.

The isolated cells are preferably cells obtained by a swab or biopsy, from the subject's own mouth. A biopsy can be obtained by using a biopsy needle under a local anesthetic, which makes the procedure quick and simple. The small biopsy core of the oral tissue can then be expanded and cultured to obtain the oral tissue cells. Cells from relatives or other donors of the same species can also be used with appropriate immunosuppression.

Methods for the isolation and culture of cells are discussed by Freshney, Culture of Animal Cells. A Manual of Basic Technique, 2d Ed., A. R. Liss, Inc., New York, 1987, Ch. 9, pp. 107-126. Cells may be isolated using techniques known to those skilled in the art. For example, the tissue can be cut into pieces, disaggregated mechanically and/or treated with digestive enzymes and/or chelating agents that weaken the connections between neighboring cells making it possible to disperse the tissue into a suspension of individual cells without appreciable cell breakage. If necessary, enzymatic dissociation can be accomplished by mincing the tissue and treating the minced tissue with any of a number of digestive enzymes either alone or in combination. These include but are not limited to trypsin, chyymotrypsin, collagenase, elastase, and/or hyaluronidase, DNase, pronase, and dispase. Mechanical disruption can also be accomplished by a number of methods including, but not limited to, scraping the surface of the tissue, the use of grinders, blenders, sieves, homogenizers, pressure cells, or insonators to name but a few.

Preferred cell types include, but are not limited to, salivary glands, submandular gland, sublingual gland, lingual glands, labial glands, buccal glands, palatine glands, striated ducts, excretory ducts, dental pulp tissue, dentin, periodontium, bone, cementum, gingival submucosa, oral submucosa, tongue and taste bud tissues. In a preferred embodiment human salivary gland cells are isolated. The oral tissue cells can be isolated from all developmental stages of the subject from fetal, neonatal, juvenile, to adult.

Once the tissue has been reduced to a suspension of individual cells, the suspension can be fractionated into subpopulations from which the cells elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting (see e.g. Freshney, (1987) Culture of Animal Cells. A Manual of Basic Techniques, 2d Ed., A. R. Liss, Inc., New York, Ch. 11 and 12, pp. 137-168). For example, salivary cells may be enriched by fluorescence-activated cell sorting. Magnetic sorting may also be used.

Cell fractionation may also be desirable, for example, when the donor has diseases such as salivary gland tumors. A salivary gland cell population may be sorted to separate tumor cells from normal noncancerous salivary gland cells. The normal noncancerous salivary gland cells, isolated from one or more sorting techniques, may then be used for salivary gland reconstruction.

Isolated cells can be cultured in vitro to increase the number of cells available for seeding into the biocompatible substrate. To prevent an immunological response after implantation of the artificial oral tissue construct, the subject may be treated with immunosuppressive agents such as, cyclosporin or FK506.

Isolated cells may be transfected with a nucleic acid sequence. Useful nucleic acid sequences may be, for example, genetic sequences which reduce or eliminate an immune response in the host. For example, the expression of cell surface antigens such as class I and class II histocompatibility antigens may be suppressed. In addition, transfection could also be used for gene delivery. Salivary gland cells may be transfected with specific genes prior to seeding onto the biocompatible substitute, such as gene for a pore forming protein through which fluid can pass, e.g., an aquaporin protein. Thus, for a salivary gland disorder, the cultured salivary gland cells can be engineered to express gene products that would produce more saliva.

The invention also relates to engineering non-fluid producing ductal cells. These non-fluid producing ductal cells, as well as other non-fluid producing cells, can even be engineered into making saliva. Unlike acinar cells, ductal cells frequently are not destroyed by irradiation. Thus, the methods of the invention can be used to genetically re-engineer ductal cells into fluid producers by giving them a gene for example, an aquaporin protein. The aquaporin protein is from the recently discovered family of proteins that form pores in cell membranes through which fluid can pass. The aquaporin gene can be inserted into an altered adenovirus and then transferred into the engineered oral tissue to produce fluid.

The biocompatible substrate comprising the salivary gland cells which express the active gene product, could be implanted into an individual who is deficient for that product. For example, genes that increase the production of saliva, e.g., aquaporin. The level of gene activity may be increased by either increasing the level of gene product present or by increasing the level of the active gene product.

The oral tissue cells grown on the substrate may be genetically engineered to produce gene products beneficial to implantation, e.g., anti-inflammatory factors, e.g., anti-GM-CSF, anti-TNF, anti-IL-1, and anti-IL-2. Alternatively, the oval tissue cells may be genetically engineered to “knock out” expression of native gene products that promote inflammation, e.g., GM-CSF, TNF, IL-1, IL-2, or “knock out” expression of MHC in order to lower the risk of rejection.

Methods for genetically engineering cells with retroviral vectors, polyethylene glycol, or other methods known to those skilled in the art can be used. These include using expression vectors which transport and express nucleic acid molecules in the cells. (See Geoddel; Gene Expression Technology: Methods in Enzymology 185, Academic Press, San Diego, Calif. (1990).

Vector DNA is introduced into prokaryotic or eukaryotic cells via conventional transformation or transfection techniques. Suitable methods for transforming or transfecting host cells can be found in Sambrook et al. Molecular Cloning: A Laboratory Manual, 2nd Edition, Cold Spring Harbor Laboratory press (1989), and other laboratory textbooks.

Once seeded onto the biocompatible substrate, the salivary gland cells will proliferate and develop on the substrate to form a salivary gland tissue layer. During in vitro culturing, the salivary gland cells develop and differentiate to produce a primitive salivary system which is capable of developing into a mature salivary system and produces saliva. The salivary gland tissue layer may also have a primitive secretory system which is capable of developing into a mature secretory system and functions to secrete saliva from the tissue engineered salivary gland. Importantly, because the biocompatible substrate has an infra-structure that permits culture medium to reach the salivary gland layer, the cell population continues to grow, divide, and remain functionally active to develop into a salivary gland that has a morphology which resembles the analogous structure in vivo.

It is important to recreate, in culture, the cellular microenvironment found in vivo for the particular oral tissue being engineered. By using a substrate that retains an infra-structure that is similar or the same as an in vivo oral tissue structure, the optimum environment for cell-cell interactions, development and differentiation of cell populations, is created.

Growth factors and regulatory factors can be added to the media to enhance, alter or modulate proliferation and cell maturation and differentiation in the cultures. The growth and activity of cells in culture can be affected by a variety of growth factors such as growth hormone, somatomedins, colony stimulating factors, erythropoietin, epidermal growth factor, hepatic erythropoietic factor (hepatopoietin), and like. Other factors which regulate proliferation and/or differentiation include prostaglandins, interleukins, and naturally-occurring chalones.

The artificial oral tissue constructs, e.g., salivary glands of the invention can be used in a variety of applications. For example, the artificial oral tissue constructs can be implanted into a subject. Implants, according to the invention, can be used to replace or augment existing tissue. For example, to treat a subject with an oral disorder e.g., a salivary gland disorder by replacing the dysfunctional oral tissue e.g., a dysfunction salivary gland of the subject with an artificial salivary gland. The subject can be monitored after implantation of the artificial salivary gland, for amelioration of the salivary gland disorder, and the production and secretion of saliva.

The artificial oral tissue can be used in vitro to screen a wide variety of compounds, for effectiveness and cytotoxicity of pharmaceutical agents, chemical agents, growth/regulatory factors. The cultures can be maintained in vitro and exposed to the compound to be tested. The activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and differential cell counts. This may be accomplished using standard cytological and/or histological techniques including the use of immunocytochemical techniques employing antibodies that define type-specific cellular antigens. The effect of various drugs on normal cells cultured in the artificial oral tissue may be assessed.

Other embodiments and used of the invention will be apparent to those skilled in the art from consideration of the specification and practice of the invention disclosed herein. All U.S. Patents and other references noted herein for whatever reason are specifically incorporated by reference. The specification and examples should be considered exemplary only with the true scope and spirit of the invention indicated by the following claims.

### EXAMPLES

### Example 1

### Methods and Materials for Tissue Engineered Salivary Glands

(i) Cell culture

Normal human salivary gland tissue was obtained during routine surgery. Informed consent was obtained from each patient prior to tissue collection and was approved by the Investigational Review Board. The tissue specimens were processed by the tissue explant technique. Briefly, the tissue was cut into I mm sized fragments, plated on culture dishes, and placed in serum-free keratinocyte growth medium (Keratinocyte SFM, Gibco, Grand Island, N.Y.) containing 5 ng/mL epidermal growth factor and 50 μg/ml bovine pituitary extract. The cells were incubated and grown in a humidified atmosphere chamber containing 5% CO₂ and maintained at 37° C.

(ii) Polymers

Unwoven sheets of polyglycolic acid polymers (density 58 mg/cc) sized 1.0×1.0×0.3 cm were used as cell delivery vehicles. Non-woven polymer meshes were composed of fibers of 15 μm in diameter with greater than 95% porosity prior to seeding. The biodegradable polymer scaffold was designed to degrade via hydrolysis in 6-8 weeks. The polymers were sterilized in ethylene oxide and stored under sterile conditions until cell delivery.

(iii) Implantation

Cultured cells were seeded onto polyglycolic acid polymers at a concentration of 50×10⁶ cells/cm³. A total of 64 polymer scaffolds (48 seeded with cells and 16 without cells) were implanted subcutaneously in athymic mice under inhalation anesthesia with Isoflurane. The polymer scaffolds were retrieved 2, 4 and 8 weeks after implantation for phenotypic and functional amylase.

(iv) Immunohistochemical and histological analyses

Serial sections (4 μm) of formalin fixed, paraffin embedded tissues were cut and stained with hematoxylin and eosin (H&E). Periodic acid Schiff staining was performed by oxidizing paraffin removed sections in 1% periodic acid for 5 min, rinsed in distilled water, washed in tap water for 1 min, and rinsed in distilled water. The sections were incubated with Schiff's reagent for 15 min, rinsed in distilled water, washed in tap water for 10 min and counterstained with Mayer's hematoxylin for 1 min. Immunhistochemical analyses were performed on cultured cells grown on Lab-Tek chamber slides (Nunc, Inc., Naperville, Ill.) and on the retrieved specimens using several specific antibodies. Sections were incubated with anti-human amylase (SIGMA, St. Louis, Mo.), anti-human cytokeratins AE1/AE3 (DAKO, Carpinteria, Calif.), and anti-Aquapolin 5 (Alpha Diagnostic International, Inc. Aan Antonio, Tex.) overnight at 4° C. Immunolabeling was performed using the avidin-biotin detection system and stained by DAB kit (Vector Laboratories, Inc Burlingame, Calif.). Sections were counterstained with Harris's hematoxylin.

(v) Western blot analysis

Cultured lysates were obtained by treating cells with lysis buffer for 10 min on ice. The lysis buffer was made with 150 mM NaCl, 20 mM Tris pH 7.4, 1% Triton-X and a protease inhibitor cocktail (Sigma Chemical Co., St Louis, Mo.). For the analysis of the cell seeded scaffolds, the implants were retrieved, homogenized in liquid nitrogen, and cell lysates were obtained. 15 μg of protein was loaded and separated on 12% (Aquaporin 5), 10% (cytokeratins AE1/AE3) and 10% (amylase) SDS-polyacryl amide gel and blotted onto Hybond ECL nitrocellulose membranes (Amersham Biotech, Buckinghamshire, England). After blocking, membranes were probed with anti-human amylase (SIGMA, St. Louis, Mo.), anti-human cytokeratins AE1/AE3 (DAKO, Carpinteria, Calif.), and anti Aquaporin 5 (Alpha Diagnostic International, Inc. Aan Antonio, Tex.). Membranes were probed by HRP conjugated secondary antibody and visualized by enhanced chemiluminescence (Perkin Elmer Life Sciences, Inc, Boston, Mass.).

(vi) RT-PCR

Total RNA was extracted from retrieved PGA scaffolds and cultured cells using RNAse kit (QIAGEN, Valencia, Calif.) according to the manufacture's instruction. 1 μg of total RNA was reverse transcribed with random primers and Superescript II (Invitrogen, Carlsbad, Calif.). PCR was performed using the primers for the human gene encoding s-Amylase (forward, 5′-AATTGATCTGGGTGGTGAGC-3′ (SEQ ID NO: 1); reverse, 5′-CTTATTTGGCGCCATCGATG-3′) (SEQ ID NO: 2) (Hokari et al. (2002) Clinica Chimica Acta 322:113-116), AQP5 (forward, 5′-CCTGTCCATTGGCCTGTCTGTCAC-3′ (SEQ ID NO: 3); reverse, 5′-GGCTCATACGTGCCTTTGATGATG-3′) (SEQ ID NO: 4) (Wang et al. (2003) Calcif Tissue Int. 72:222-227) and GAPDH (forward, 5′-GGAAGGTGAAGGTCGGAGTC-3′ (SEQ ID NO: 5); reverse, 5′-CAGTAGAGGCAGGGATGATG-3′) (SEQ ID NO: 6). Standard PCR conditions were used with annealing at 65° C. (amylase and AQP5) and 50° C. (GAPDH).

(vii) Biochemical Assay

The retrieved engineered salivary gland tissues were placed on Petri dishes with culture medium at 37° C. for 72 hours. The culture medium was collected from primary salivary gland cells and from the retrieved engineered tissues after 72 hours of incubation for α-amylase activity assays. The culture medium from fibroblasts, implants without cells and host subcutaneous tissues served as controls. 25 μl of medium from each sample was mixed with 1 ml of chromogenic amylase substrate (2-chloro-p-nitrophenol linked with maltotriose) at 37° C. (BIOTRON Diagnostic Inc. Hemet, Calif.). Amylase activities were measured with a spectrophotometer at 405 nm. The absorbance was kinetically measured at each 30 sec for 2 min at 37° C. and the amylase activity was calculated according to the manufacturer's instructions.

### Example 2

### Production of Tissue Engineered Salivary Glands

This example describes how to produce functional salivary gland tissue using the methods of the invention. Salivary gland cells, e.g., glandular epithelial cells, were isolated and expanded, as described in Example 1. The expanded cells were seeded onto a PGA matrix and allowed to attach and grow before implantation. The salivary gland constructs were then implanted subcutaneously in athymic mice and retrieved 2, 4 and 8 weeks after implantation for phenotypic and functional analyses.

The results showed that primary salivary gland cells were successfully isolated from the tissue, grown and expanded in culture. The cells retained their phenotypic and functional expression at each stage of subculture throughout the entire study period expressing α-amylase, aquaporin5, and cytokeratins AE1/AE3 (data not shown). Immunocytochemical studies using anti human cytokeratins AE1/AE3 and anti human amylase antibodies stained the glandular cells positively. Aquaporin 5 was expressed only on the fully differentiated cells in culture.

At retrieval, the polymer scaffolds with the salivary gland cells formed tissue structures in all instances. Formation of multiple vascular structures supplying the cell seeded implants was evident grossly. There was no evidence of inflammation, infection or fluid collection at the implant sites.

Characterization of the tissue engineered salivary glands was performed as described in Example 1. Histologically, acinar-like structures were observed in the scaffolds seeded with salivary gland cells by 2 weeks after implantation. The cells retained their phenotypic expressions (data not shown). The cells were analyzed with hematoxylin-eosin, and periodic acid schiff staining. Periodic acid Schiff staining demonstrated the presence of mucin in the cell implanted tissues. The cells forming the acinar-like structures expressed human cytokeratins AE1/AE3, Aquaporin 5 and ex-amylase, as confirmed by immunohistochemistry using cell specific antibodies. Generous angiogenesis was observed in all of the cell seeded implants. There was no evidence of glandular-like structures formed in any of the control scaffolds without cells. None of the control scaffolds expressed human cytokeratins AE1/AE3, Aquaporin 5 or amylase.

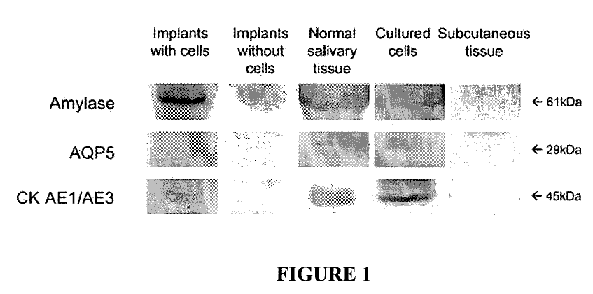

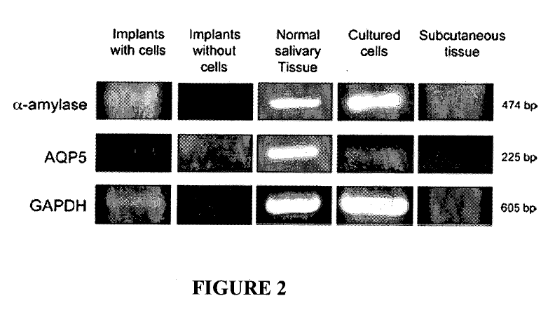

Western blot analyses of the primary human salivary gland cells and implants with cells showed expression of α-amylase (61 kDa), Aquaporin 5 (29 kDa) and cytokeratins AE1/AE3 (45 kDa), which were similar to normal human salivary gland tissue (FIG. 1). Polymer implants without cells and host subcutaneous tissue failed to express these proteins. Expression of α-amylase and Aquaporin 5 mRNA was detected and analyzed. RT-PCR products of expected size of 474 bp (α-amylase) and 225 bp (Aquaporin 5) were obtained with RNA from primary human salivary gland cells, implants with cells and normal human salivary gland tissues (FIG. 2). There was no detection of these genes in the scaffolds without cells and host subcutaneous tissues.

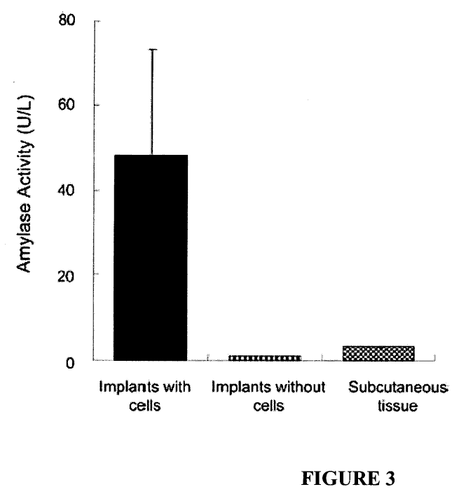

Consistent levels of amylase activities were detected in the cultured salivary gland cell medium at all subculture stages (>7 subcultures; 11.2 U/L). Culture medium incubated with fibroblasts failed to show any amylase activity. High levels of amylase activity of the medium incubated with implanted scaffolds with cells were detected. The implants without salivary gland cells and host subcutaneous tissues showed basal levels of activity (FIG. 3).

These results show that salivary gland cells can be expanded in culture and are able to maintain their phenotypical and functional characteristics. These cultured salivary glands can be used to engineer functional salivary gland tissue, which secrete consistent levels of α-amylase in vivo.

One of the basic components required to engineer functional tissues is the cell. Development of a reliable cell expansion system has been one of the most important challenges in tissue engineering. Using defined culture systems, it is possible to expand cells from a single 1 cm² tissue specimen to a surface area of 4,204 m² within 2 months (Oberpenning et al. (1999) Nat Biotechnol 17:149-155; and Cilento et al. (1994) J Urol 152:665-670). The same strategy was used for salivary gland cells. The cells were successfully isolated from salivary gland tissue, grown and expanded in large quantities, and implanted in vivo to achieve functional salivary gland tissue.

Salivary glandular cells expanded in culture retained their phenotypic and functional characteristics during the course of the study. Phenotypic expression of salivary glandular cells was confirmed histologically, immunocytochemically, and with Western blot analyses using cell specific antibodies. Gene expression and cellular function were further confirmed by RT-PCR and amylase activity assays. These cellular characteristics were retained even when the cells were introduced into the body system, indicating the feasibility of creating salivary gland tissues for functional augmentation or replacement.

The engineered salivary gland tissue did not exhibit signs of fluid accumulation at the site of implantation, possibly due to the continuous absorption of the fluid into the host circulation. Approximately 99.5% of saliva is composed of water. Aquaporin 5, a water channel protein, is known to play an important role in the main water flow pathway from the acinar cells to the lumen of the salivary gland (Ma et al. (1999) J Biol Chem 274:20071-20074 and He et al. (1997) Pflugers Arch 433:260-268). The presence of Aquaporin 5 proteins and RNA within the retrieved tissue indicates that the engineered tissue possesses salivary glandular function. Amylase is one of the elements contained in saliva and the presence of amylase protein and RNA was demonstrated within the retrieved engineered tissues. Furthermore, the secretion of amylase by the engineered tissues ex vivo in controlled chambers, was confirmed.

Another basic component involved in the engineering of viable tissues is the cell delivery vehicle. In this study, biodegradable polyglycolic acid polymers were used as the scaffold matrix. The structure of the polymer scaffolds was fabricated to accommodate a large number of cells while promoting larger nutrient diffusion distances. In addition, the polymers were designed to degrade over 6-8 weeks, which allows adequate time for the seeded cells to form viable tissues in vivo. The salivary glandular cells seeded on the scaffolds readily attached to the polymer fibers, remained viable and formed salivary gland tissue in vivo. None of the animals implanted with the polymer scaffolds exhibited untoward effects or had any evidence of inflammation or infection.

This data shows that functional salivary gland tissue can be engineered and implanted to provide a continuous supply of saliva in patients, particularly patients with radiation induced xerostomia. Theoretically, patients undergoing radiation therapy for head and neck cancer could undergo salivary gland tissue collection at the time of pre-treatment panendoscopy. This tissue could be cryopreserved and later grown, expanded and seeded on biodegradable polymer scaffolds. After completion of radiation or chemoradiation therapy, the seeded polymer scaffolds can then be implanted within the parotid bed in order to utilize the existing host ductal system. Alternatively, a ductal system could be engineered using nanotechnology to fabricate scaffolds with micro-channels. These cell seeded scaffolds can then be implanted beneath the oral mucosa and the saliva can be directed into the oral cavity via the engineered ductal system.

Collectively, these results show that primary human salivary glandular cells seeded on biodegradable polymers are able to form functional tissues in vivo. The engineered tissue, composed of glandular epithelial cells, is able to produce amylase and possesses Water channel proteins. This autologous cell based system provides a new treatment option for patients suffering from conditions leading to salivary hypofunction, such as radiation induced xerostomia. The successful engineering of functional salivary gland tissue represents a therapeutic alternative to the current poor treatment options for salivary hypofunction.

### Example 3

### Methods and Materials for the Formation of Acinar Structures

This example describes how to create acinar structures from human salivary gland epithelial cells. Normal human salivary gland tissue was obtained during routine surgery as described in Example 1.

(i) Three Dimensional Culture

Sub-confluent human salivary gland epithelial primary culture cells were trypsinized and neutralized. 1×10⁶ cells were resuspended in 1 ml of Keratinocyte SFM containing 5 ng/ml epidermal growth factor and 50 μg/ml bovine pituitary extract (Complete Medium) and kept on ice. The following steps were conducted on ice unless indicated. Neutralized collagen solution comprising 1800 μl of Rat Tail collagen type I (Roche Applied Science) and 200 μl of Medium 199 10 × (GIBCO) were mixed well and neutralized by adding 1N NaOH solution. The following three different ratios of collagen based mixture gels were prepared. Cell suspension : Neutralized Collagen Solution: Growth Factor Reduced Matrigel at a ratio of (i) 25:75:0, (ii) 25:60:15, and (iii) 25:45:30. 150μl/well of each mixture as plated into a 48 well dish. The gelling process was conducted by incubating the dish in a humidified atmosphere chamber containing 5% CO₂ at 37° C. for 20 min. 250 μl/well of compete medium was added to each well and incubated. The medium was changed every two days. The cells were observed under the phase contrast microscope and recorded by digital capture system.

(ii) Immunohistochemical Analysis

For immunohistochemical analysis, primary antibodies: Anti-human occluding and Anti-human ZO-1 were obtained from Zymed. Anti-human Aquaporin 5 was obtained from Alpha Diagnostic International Inc., San Antaonio, Tex.). Anti-human alpha amylase was obtained from Sigma, St, Louis Mo. Secondary antibody, Anti-Rabbit Fluorescence was obtained from Vector Laboratory.

The three dimensional gels were cultured with cells for 7 days, fixed in 4% paraformaldehyde (EM Sciences, Fort Washington, Pa.) for 30 min at room temperature (RT), and washed with PBS. Gels were equilibrated in equilibration solution (20 mM glycine, 75 mM NH₄Cl, 0.1% Triton X-100, in PBS w/o Ca²⁺ Mg¹⁺) for 30 min at RT to reduce later fluorescein background fluorescence from extracelluar matrix gel. The gels were then preincubated for 1 h at 4° C. in blocking buffer (0.05% Triton X-100, 0.7% Fish gelatin in PBS). Primary antibodies were diluted in blocking buffer and incubation with the gels was performed for 36 h at 4° C. on a rocking stage. For control samples, the primary antibody was omitted. Three washes with washing solution (0.05% Triton X-100 in PBS) were performed over 24 h at 4° C. The gels were then incubated with secondary antibodies in blocking buffer were incubated for 24 h at 4° C. Three washes with washing solution over a 24-h period were performed to remove any unbound secondary antibody from the gel. Samples were then mounted with Fluoromount (Southern Biotechnology Associates Inc, Birmingham, Ala.). Evaluation of the stained cells was performed by phase contrast microscope and scanning laser confocal microscopy (Zeiss, LSM-510) equipped with an argon/krypton laser and oil-DIC objectives. The images were scanned at 1024×1024 or 2048×2048 pixels in multitracking mode alternating the excitations for FITC/Cy2. Serial confocal images were collected using the same laser energies and magnifications. Images were processed with Photoshop software (Adobe).

(iii) Reverse-Transcription Polymerase Chain Reaction (RT-PCR)

Total RNA was extracted from each gel on day 1, 3, 5, and 7 using RNeasy kit (Qiagen, Valencia, Calif.) according to the manufacture's instruction. One microgram of total RNA was reverse transcribed with random primers and Superescript II (Invitrogen, Carlsbad, Calif.) into cDNA. PCR was performed using the manufacturer's recommended conditions (Takara; Siga) using the following primers: occludin (sense 5-TCAGGGAATATCCACCTATCACTTCAG-3 (SEQ ID NO: 7) and antisense 5-CATCAGCAGCAGCCATGTACTCTTCAC-3 (SEQ ID NO: 8), amplicon length 136 bp); ZO-1 (5-CGGTCCTCTGAGCCTGTAAG-3 (SEQ ID NO: 9) and antisense 5-GGATCTACATGCGACGACAA-3 (SEQ ID NO: 10), amplicon length 435 bp); salivary-amylase (sense 5′-AATTGATCTGGGTGGTGAGC-3 (SEQ ID NO: 11) and antisense 5′-CTTATTTGGCGCCATCGATG-3′) (SEQ ID NO: 12), aquaporin5 (sense 5′-CCTGTCCATTGGCCTGTCTGTCAC-3′ (SEQ ID NO: 13) and antisense 5′-GGCTCATACGTGCCTTTGATGATG-3′) (SEQ ID NO: 14); GAPDH (sense 5′-GGAAGGTGAAGGTCGGAGTC-3′ (SEQ ID NO: 15) and antisense 5′-CAGTAGAGGCAGGGATGATG-3′) (SEQ ID NO: 16). Conditions applied for PCR were 96° C. for 30 sec, 30 cycles of 96° C. for 15 sec, 55° C. (occludin and ZO-1), 65° C. (amylase and aquaporin5), 50° C. (GAPDH)) for 30 sec, 72° C. for 1 min, and 72° C. for 7 min. Total PCR reaction was analyzed by electrophoresis impregnated 1% agarose gels.

### Example 4

### Production of Acinar Structures in Three-Dimensional Gel Substrates

By using the methods described in Example 3, acinar reconstruction in three dimensional culture system was achieved. The concentration of collagen was determined from several different ranges. In order to distribute seeded cells uniformly in the collagen based matrix, a ratio of cell solution : matrix solution of 1:3 per gel, was used.

When human salivary gland epithelial primary culture cells were cultured in collagen based three dimensional culture system, each single cell started to reconstitute its acinar formation. By day 2, single cells had divided into 2 to 4 cells. On day 4, some of the acinar-like structures were observed in collagen gel three dimensional culture system. Those acinar-like structures were predominant in the collagen gel by day 6.

Modifications of the three-dimensional gel matrix provided insight into improving acinar and ductal formation. In particular, matrigel added gel provided improved conditions to reconstitute of acinar and ductal formation.

The collagen type I based three dimensional culture system was also modified and optimized this by mixing it with growth factor reduced matrigel. Matrigel was mixed at the related ratio to collagen type I at 0%, 20% and 40%. The results demonstrated that the boundary of each of the cells among the acinar structure seemed to be isolated from cell to cell in the absence of matrigel. The boundary of each of the cells also appeared to be more flexible, fitting and contacting into the large area to next to the cells in matirgel added gel. This was observed when higher concentration of matirgel was added to the gel. In addition to the acinar structure, many ductul-like structures were also reconstituted and formed a network between acinar-like structures by day 16 in the 30% matrigel added gel.

Functional analysis and immunohistochemistry characterization of the reconstituted acinar structures in three dimensional culture system revealed the presence of amylase positive cells, and AQP5 positive. The presence of tight junction proteins was also investigated, and the cells were positive for ZO-1. Occludin and claudin-1 tight junction protein are still under investigation.

Collectively, these results show that each acinar-like structure could be reconstituted from single human primary cultured salivary gland epithelial cell in collagen type I based three dimensional culture, with maintaining tight junction formation, water channel protein expression and amylase productivity.

The collagen based gel scaffold provided a suitable system to investigate the detailed process of acinar recantation. This provides a system that allows the acinar structures to be reconstituted in vitro to optimize tissue reconstruction in vivo. Each acinar like structure can be reconstituted from a single cell. If the distance between two individual cells is close enough, the cells try to reach each other by growing processes toward the other cell. After two days from seeding, the light path refraction was observed in a concentric circle area surrounding the cell. This phenomenon may have been induced by extra matrix modification around cell. Cells remodel their surrounding matrix by secreting protease and producing new extracellular matrix modifing surrounded growth matrix. In same way some signaling proteins may also be secreted from cells, spread and reach to the other cells by diffusion in collagen based matrix.

These results further demonstrate that by using collagen based amorphous gel for cell reconstitution, the gel may provide a more flexible environment which allows cells to proliferate and reconstitute in all directions rather than a single direction, as is often the case with in PGA sheet matrices. As PGA sheets consists of narrow fibers, cells seeded on them may have to attach on the surface of fibers and then proliferate along the fibers. As the fibers degrade over a period of weeks, more cell-cell contact occurs. However, use of the three-dimensional gels as a substrate for cell growth permits the cells to grow in all dimensions early on. Thus, for cells that develop structural cellular features within a few days after seeding, this method offers an alternative. This is particularly important for acinar reconstitution, which was accomplished within 6 days in vitro within the three-dimensional collagen based gel substrate. The collagen gel matrix and cell mixture can be injected into native parotid or submandibular tissue, and the implanted cells can integrate with native tissue. Therefore, collagen based gels are suitable scaffolds for salivary gland reconstitution.

### Example 5

### Role of Extracellular Matrix Proteins in Three-Dimensional Gel Substrates

The following example examines how extracellular matrix proteins affect reconstitution in three-dimensional gel substrates. Extracellular matrix proteins play very important role for cells behavior. Examples 3-4 describe how to reconstitute a functional subunit in three-dimensional gel substrate using autologous cells. In this example, the impact of collagen type I and growth factor on primary cultured human salivary gland epithelial cells during reconstitution of acinar and ductal formation in the three-dimensional gel matrix were investigated. The following three different matrix compositions were used: (i) collagen type I only, (ii) collagen co-mixed with 15% matrigel, and (iii) collagen co-mixed with 30% matrigel.

Within two days of incubation, the cell started to divide into two cells. The results (not shown) demonstrated that the behavior of the cells was different under each condition. In the collagen type I gel, each divided cells remains in contact with the other with only a small area of each cell wall maintaining contact. Each cell retains their round cell shape. The cells continue to proliferate and by six days, the cells form a spherical cluster of cells, with each cell of the cluster retaining its round cell shape.

With cells grown in the collagen co-mixed with 30% matrigel, the divided cells remain in contact with each other, but adjust their cell shape to fit together and form tight contacts along a large area of each cell wall. Eventually cells formed acinar-like structures maintaining those cells relationship.

In cells grown in the collagen co-mixed with 15% matrigel, both of the above two different processes were observed. In addition, the individual acinar-like structures started to develop network via duct like structures in 15% matrigel by day 16.

### Example 6

### Tight Junction and Acinar Functions

This example investigates tight junction and acinar functions of salivary gland cells grown in a three-dimensional gel substrate. Tight junctions form a barrier to diffusion of molecules from lumen to the tissue parenchyma (barrier function) and restricts the diffusion of lipids and proteins between the apical and basolateral plasma membrane (fence function) in epithelial cells and endothelial cells. Tight junctions proteins comprise occludin, claudins, janctinal adhesion molecule (JAM), ZO family and other tight junction associated proteins. Tight junction permeability is regulated by integration of those tight junction proteins and cell signaling systems (Harhaj et al. (2004) Int. J Biochem Cell Biol. 36:1206-37). ZO plays central role in orchestrating tightjunction complex. Occludin has extracellular loop domain and recruited by ZO to localize in tight junction. Occludin is not required to maintain the structural integrity of tight junction (Saitou et al. (1998) J Cell Biol. 141:397-408), however, lack of occludin exhibits apparent abnormalities in testis and salivary gland, thinning of compact bone, calcium deposits in brain and gastric epithelium using knockout mice study (Saitou et al. (2000) Mol Biol Cell. 11:4131-42). The function of salivary gland is to secrete saliva which contained several proteins but 99.5% is water. Water transportation in salivary glad acina is conducted through paracellular flux and transcellular water movement (Baum (1993) Ann N Y Acad Sci. 694:17-23). Aquaporin 5 water channel protein plays an important role for transcellular water transportation (Song (1999) J Biol Chem. 274:20071-4). In this study, acinar structures were reconstituted from human primary cultured salivary gland epithelial cells. These structures maintained the expression of tight junction proteins occludin and ZO-1, the water channel protein, aquaporin 5, and amylase.

Collectively, these studies show that each acinar structure can be reconstituted from each single human salivary gland epithelial primary cultured cell within a collagen-based three-dimensional culture system. The reconstituted cells maintain their functional characteristic such as amylase production, water channel protein (aquaporin 5) production, and tight junction proteins (occludin and ZO-1) production. The extracellular matrix protein affects the formation of acinar structures by influencing tight junction protein expression. Accordingly, every human salivary gland epithelial cell retrieved from patient has the potential to develop into a salivary gland unit by reconstituting acinar-like structures and ductal-structures.

What is claimed is:

1. A method of preparing an artificial oral tissue construct comprising:
seeding a population of cultured oral tissue cells onto a substrate, such that the oral tissue cells attach to the substrate; and
culturing the oral tissue cells on the substrate until the oral tissue cells produce an oral tissue layer comprising a primitive salivary system.

2. The method of claim 1, wherein the substrate is a biocompatible substrate.

3. The method of claim 2, wherein the biocompatible substrate is selected from the group consisting of cellulose ether, cellulose, cellulosic ester, fluorinated polyethylene, poly-4-rethylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends thereof.

4. The method of claim 2, wherein the biocompatible substrate is polyglycolic acid.

5. The method of claim 1, wherein the artificial oral tissue construct further comprises a primitive secretory system.

6. The method of claim 1, wherein the artificial oral tissue construct is selected from the group consisting of salivary glands, submandular gland, sublingual gland, lingual glands, labial glands, buccal glands, palatine glands, striated ducts, and excretory ducts.

7. The method of claim 1, wherein the artificial oral tissue construct is a salivary gland.

8. The method of claim 1, wherein the oral tissue cells are human salivary gland cells.

9. A method of preparing an artificial salivary gland construct comprising:
seeding a population of cultured salivary gland cells onto a substrate, such that the salivary gland cells attach to the substrate; and
culturing the salivary gland cells on the substrate until the salivary gland cells produce a salivary gland tissue layer comprising a primitive salivary system.

10. The method of claim 9, wherein the substrate is a biocompatible substrate.

11. The method of claim 10, wherein the biocompatible substrate is selected from the group consisting of cellulose ether, cellulose, cellulosic ester, fluorinated polyethylene, poly-4-rethylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, ureaformaldehyde, or copolymers or physical blends thereof.

12. The method of claim 10, wherein the biocompatible substrate is polyglycolic acid.

13. The method of claim 9, wherein the artificial salivary gland construct further comprises a primitive secretory system.

14. The method of claim 9, wherein the salivary gland cells are human salivary gland cells.

15. An artificial oral tissue construct comprising a substrate seeded with a population of cultured oral tissue cells, wherein the oral tissue cells attach to the substrate to produce an oral tissue layer comprising a primitive salivary system.

16. The artificial oral tissue construct claim 15, wherein the substrate is a biocompatible substrate.

17. The artificial oral tissue construct of claim 16, wherein the bioconipatible substrate is selected from the group consisting of cellulose ether, cellulose, cellulosic ester, fluorinated polyethylene, poly-4-rethylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends thereof.

18. The artificial oral tissue construct of claim 16, wherein the biocompatible substrate is polyglycolic acid.

19. The artificial oral tissue construct of claim 15, further comprising a primitive secretory system.

20. The artificial oral tissue construct of claim 15, wherein the construct is selected from the group consisting of salivary glands, submandular gland, sublingual gland, lingual glands, labial glands, buccal glands, palatine glands, striated ducts, and excretory ducts.

21. The artificial oral tissue construct of claim 15, wherein the construct is a salivary gland.

22. The artificial oral tissue construct of claim 15, wherein the oral tissue cells are human salivary gland cells.

23. An artificial salivary gland construct comprising a substrate seeded with a population of cultured salivary gland cells, wherein the salivary gland cells attach to the substrate to produce a salivary gland tissue layer comprising a primitive salivary system.

24. The artificial salivary gland construct of claim 23, wherein the substrate is a biocompatible substrate.

25. The artificial salivary gland construct of claim 24, wherein the biocompatible substrate is selected from the group consisting of biocompatible substrate is selected from the group consisting of cellulose ether, cellulose, cellulosic ester, fluorinated polyethylene, poly-4-rethylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends thereof.

26. The artificial salivary gland construct of claim 24, wherein the biocompatible substrate is polyglycolic acid.

27. The artificial salivary gland construct of claim 23, further comprising a primitive secretory system.

28. The artificial salivary gland construct of claim 23, wherein the salivary gland cells are human salivary gland cells.

29. A method of ameliorating an oral disorder in a subject comprising:
implanting a biocompatible substrate seeded with a population of cultured oral tissue cells, wherein the oral tissue cells attach to the biocompatible substrate to produce an oral tissue layer comprising a primitive salivary system; and
monitoring the subject for the amelioration of the oral disorder.

30. The method of claim 29, wherein the oral disorder is selected from the group consisting of salivary gland tumors, cystic fibrosis, Sjögren's syndrome, sialoadenitis, parotitis, sialoangitis, sialodochitis, sialolithiasis, sialodocholithiasis, mucocele, ranula, hyposecretion, ptyalism, sialorrhea, xerostomia, benign lymphoepithelial lesion of salivary gland; sialectasia; sialosis; stenosis of salivary duct; and stricture of salivary duct.

31. The method of claim 28, wherein the oral tissue cells are human salivary gland cells.

32. A method of ameliorating xerostomia in a subject comprising:
implanting a biocompatible substrate seeded with a population of cultured salivary gland cells, wherein the salivary gland cells attach to the biocompatible substrate to produce a salivary gland tissue layer comprising a primitive salivary system; and
monitoring the subject for the amelioration of xerostomia.

33. The method of claim 32, wherein the oral tissue cells are human salivary gland cells.

34. A method of preparing an artificial oral tissue construct comprising:
seeding a population of cultured oral tissue cells onto a three-dimensional substrate, such that the oral tissue cells attach into the substrate; and
culturing the oral tissue cells in the substrate until the oral tissue cells produce acinar-like structures.

35. The method of claim 34, wherein the cells further produce ductal-like structures.


SEQUENCE LISTING (16 sequences)

SEQ ID NO: 1 - DNA, 20 bp, Artificial
  SYNTHETIC CONSTRUCT
aattgatctgggtggtgagc

SEQ ID NO: 2 - DNA, 20 bp, Artificial
  SYNTHETIC CONSTRUCT
cttatttggcgccatcgatg

SEQ ID NO: 3 - DNA, 24 bp, Artificial
  SYNTHETIC CONSTRUCT
cctgtccattggcctgtctgtcac

SEQ ID NO: 4 - DNA, 24 bp, Artificial
  SYNTHETIC CONSTRUCT
ggctcatacgtgcctttgatgatg

SEQ ID NO: 5 - DNA, 20 bp, Artificial
  SYNTHETIC CONSTRUCT
ggaaggtgaaggtcggagtc

SEQ ID NO: 6 - DNA, 20 bp, Artificial
  SYNTHETIC CONSTRUCT
cagtagaggcagggatgatg

SEQ ID NO: 7 - DNA, 27 bp, Artificial
  SYNTHETIC CONSTRUCT
tcagggaatatccacctatcacttcag

SEQ ID NO: 8 - DNA, 27 bp, Artificial
  SYNTHETIC CONSTRUCT
catcagcagcagccatgtactcttcac

SEQ ID NO: 9 - DNA, 20 bp, Artificial
  SYNTHETIC CONSTRUCT
cggtcctctgagcctgtaag

SEQ ID NO: 10 - DNA, 20 bp, Artificial
  SYNTHETIC CONSTRUCT
ggatctacatgcgacgacaa

SEQ ID NO: 11 - DNA, 20 bp, Artificial
  SYNTHETIC CONSTRUCT
aattgatctgggtggtgagc

SEQ ID NO: 12 - DNA, 20 bp, Artificial
  SYNTHETIC CONSTRUCT
cttatttggcgccatcgatg

SEQ ID NO: 13 - DNA, 24 bp, Artificial
  SYNTHETIC CONSTRUCT
cctgtccattggcctgtctgtcac

SEQ ID NO: 14 - DNA, 24 bp, Artificial
  SYNTHETIC CONSTRUCT
ggctcatacgtgcctttgatgatg

SEQ ID NO: 15 - DNA, 20 bp, Artificial
  SYNTHETIC CONSTRUCT
ggaaggtgaaggtcggagtc

SEQ ID NO: 16 - DNA, 20 bp, Artificial
  SYNTHETIC CONSTRUCT
cagtagaggcagggatgatg